(19) United States
(12) Patent Application Publication
(10) Pub. No.: US 2024/0307320 A1
(43) Pub. Date: Sep. 19, 2024

(54) A System and Method for Measuring and Stimulating Autophagy

(71) Applicant: Biophagy, Inc., Albuquerque, NM (US)

(72) Inventors: Mary Ortner, Mountainair, NM (US); Stuart Rose, Albuquerque, NM (US); Ian Henderson, Sandia Park, NM (US)

(21) Appl. No.: 18/577,088

(22) PCT Filed: Jul. 8, 2022
(86) PCT No.: PCT/US2022/036495

§ 371 Date: Jan. 5, 2024

Related U.S. Application Data
(60) Provisional application No. 63/219,469, filed on Jul. 8, 2021.

(51) Int. Cl. A61K 31/05; A61B 10/02; A61K 31/12; A61K 31/132; A61K 31/192; A61K 31/352; A61K 31/4412; A61K 31/7016

(52) U.S. Cl. CPC A61K 31/05; A61B 10/02; A61K 31/12; A61K 31/132; A61K 31/192; A61K 31/352; A61K 31/4412; A61K 31/7016; A61B 2010/0216

(57) ABSTRACT

A system for stimulating autophagy comprising a sample collection kit (either a buccal swab kit or a lancet kit) and one or more compounds that stimulate autophagy. The buccal swab kit has at least one buccal swab, a collection container, a storage device, a shipping package, a set of instructions, and at least one set of gloves. The lancet kit has at least one sterile lancet or an automated lancet device for capillary bloodletting, a sealed packet with an isopropyl alcohol wipe or pad, a collection container, a storage device, and a shipping package. A method of stimulating autophagy in an individual comprising the steps of providing the individual with at least one sample collection kit, testing the sample provided by the individual to determine the individual's level of autophagy, and administering one or more compounds to the individual to stimulate autophagy on a single use or subscription basis.

### CROSS-REFERENCE TO RELATED APPLICATIONS

This PCT application claims priority to U.S. Provisional Patent Application No. 63/219,469, titled “A System and Method for Measuring and Stimulating Autophagy,” filed Jul. 8, 2021, the contents of which are incorporated by reference in their entirety herein.

### BACKGROUND

Autophagy is the body's way of cleaning out damaged/denatured material and infectious agents from cells in order to maintain cellular health; however, the level of autophagy decreases as a person ages.

Accordingly, there is a need for a system and method of measuring and stimulating autophagy.

### SUMMARY

The present invention addresses this need. In a first embodiment, the present invention is directed to a system for stimulating autophagy. The system comprises a sample collection kit, and one or more compounds that stimulate autophagy. The sample collection kit can be a buccal swab kit or a lancet kit.

The buccal swab kit has at least one buccal swab, at least one collection container, at least one pre-labeled storage device, at least one pre-labeled, pre-paid shipping package, a set of instructions on how to use the buccal swab, and at least one set of gloves. The storage device can be pre-labeled with a bar code or QR code designating the individual client and other related information.

The lancet kit has at least one sterile lancet or an automated lancet device for capillary bloodletting, a sealed packet with an isopropyl alcohol wipe or pad, a collection container for receiving a blood sample procured using the lancet or lancet device, a storage device for storing the collection container containing the blood sample, and a pre-labeled, pre-paid shipping package for return to the company for further processing.

Optionally, both kits have a set of instructions on how to use the buccal swab, lancet or lancet device, and how to return the sample to the company. The collection container for the blood sample is a paper filter or other solid material for absorbing the blood sample.

The storage device can be pre-labeled with a bar code or QR code designating the individual client and other related information.

The one or more compounds or related natural product extracts or dry materials are selected from the group consisting of: resveratrol, curcumin/turmeric, spermidine, fisetin, beberine, quercetin, ginger, rutin, black pepper, oridonin, evodiamine, epigallocatechin gallate (EGCG), theanine, citrus bergamont, herbs (for example but not limited to oregano, sage, rosemary, etc.), isoliquiritigenin, alpha-magostin, wogonin, boswellia, leuteolin, piperlongumine, pterostilbene, wheat germ extract, bladderwrack extract, ginseng, anise, minerals (for example but not limited to, zinc, manganese, etc.), grape seed extract, R-alpha lipoic acid, soy lipid extract, trehalose, caffeic acid, and salicin.

In a second embodiment, the present invention is directed to a method of stimulating autophagy in an individual, the method comprising the steps of: a) providing the individual with at least one sample collection kit whereby the individual collects a sample; b) testing the sample provided by the individual to determine the individual's level of autophagy; and c) administering one or more compounds to the individual to stimulate autophagy. The step of administering can comprise administering the compounds via a subscription program.

In a third embodiment, the present invention is directed to a combination of compounds for stimulating autophagy wherein the combination comprises two or more compounds or related natural product extracts or dry materials selected from the group consisting of: resveratrol, curcumin/turmeric, spermidine, fisetin, beberine, quercetin, ginger, rutin, black pepper, oridonin, evodiamine, epigallocatechin gallate (EGCG), theanine, citrus bergamont, herbs (for example but not limited to oregano, sage, rosemary, etc.), isoliquiritigenin, alpha-magostin, wogonin, boswellia, leuteolin, piperlongumine, pterostilbene, wheat germ extract, bladderwrack extract, ginseng, anise, minerals (for example but not limited to zinc, manganese, etc.), grape seed extract, R-alpha lipoic acid, soy lipid extract, trehalose, caffeic acid, and salicin.

### DRAWINGS

These and other features, aspects, and advantages of the present invention will become better understood with regard to the following description, appended claims, and accompanying drawings.

FIG. 1 is a perspective view of a first embodiment of a buccal swab kit having features of the present invention;

FIG. 2 is a depiction of how to use the kit of FIG. 1;

FIG. 3 is a perspective view of a lancet kit having features of the present invention;

FIG. 4A depicts a western blot analysis of autophagy stimulation by spermidine (10 uM), fisetin (10 uM), and a combination of both compounds;

FIG. 4B is a graphical presentation of the western blot data in FIG. 4A;

FIG. 5A is a western blot analysis of autophagy stimulation by spermidine (25 uM), fisetin (25 uM), and a combination of both compounds; and

FIG. 5B is a graphical presentation of the western blot data in FIG. 5A.

### DESCRIPTION

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

As used in this disclosure, the term “comprise” and variations of the term, such as “comprising” and “comprises,” are not intended to exclude other additives, components, integers ingredients or steps.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any system, any device or part of a device disclosed in this disclosure will be determined by its intended use.

Autophagy is a process whereby cells break down and recycle denatured, tangled, and clumped materials that interfere with cellular function and health. This includes damaged organelles and invading bacteria and viruses.

Autophagy tends to decrease with age, exacerbating many related diseases, infirmities, and disabilities.

Moreover, several diseases (neurological conditions, inflammation, metabolic syndromes, cancer, etc.) are connected with unbalanced or decreased autophagy function. Accordingly, autophagy is critical for cellular health.

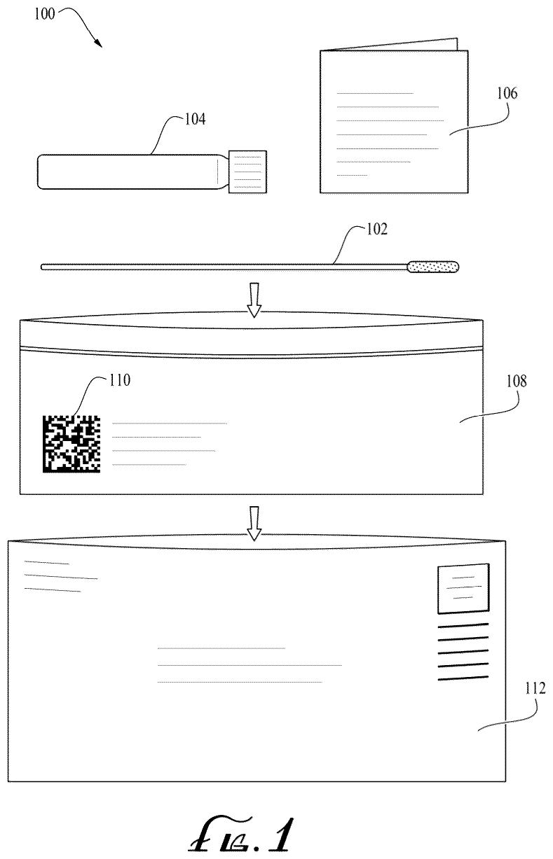

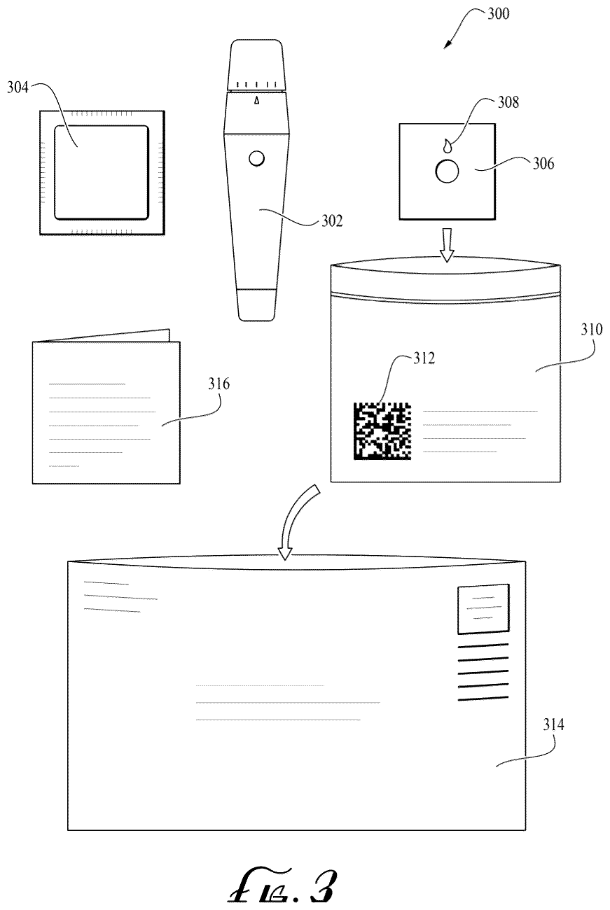

Referring now to FIGS. 1 through 3, the invention is directed to a system and method for measuring and stimulating autophagy. In a first embodiment, the system comprises a buccal kit 100 for use by an individual to obtain a buccal swab 102 of themselves (or another individual) and submit the buccal swab 102 for testing to determine that individual's own personal level of autophagy.

At an individual's discretion or based on the level of autophagy determined, the individual can then take one or more compounds to stimulate autophagy for a specified period of time, ultimately increasing their own personal level of autophagy which can then be measured using a buccal kit 100 or a lancet kit 300.

The buccal kit 100 comprises at least a buccal swab 102 and a collection container 104. The collection container 104 can be a test tube, a vial, or a sterile envelope into which the used swab is placed, instructions 106 for how to use the buccal swab 102 to obtain a sample from the individual and return the sample to the company, a storage device 108 for sealing the collection container 104 with the individual's sample inside (pre-labeled with a bar code or QR code 110 designating the individual client and other related information), and a pre-labeled, pre-paid shipping package (which can be an envelope or a box) 112 for return to the company for further processing. Optionally, the kit 100 can also include at least one set of gloves (not shown).

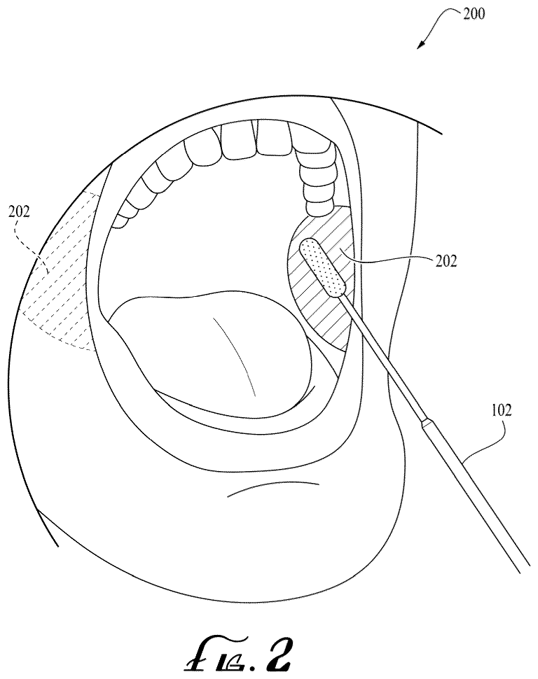

Referring now to FIG. 2, there is shown a method 200 of using the buccal kit 100. To use the kit 100, the individual firmly presses and twirls the swab 102 against the inside of their inner cheek 202 using an up and down motion from front to back and back to front. This swabbing motion should be performed for at least 30 seconds per swab 102 to collect cells. It is important to collect samples from maximum mucosal surfaces and more than one swab may be used. Once collected, the swab 102 is then placed into the collection container 104 and put into a pre-labeled storage device 108 which is shipped back to the company in a pre-paid, pre-labeled shipping package (envelope or box) 112 for testing.

Optionally, the individual can swab the inner cheek 202 of someone other than themselves, if that other person is the one being tested.

In a second embodiment, the system can comprise a lancet kit 300. The lancet kit 300 comprises at least one sterile lancet or an automated lancet device 302 for capillary bloodletting, a sealed packet with an isopropyl alcohol wipe or pad 304, a collection container 306 for receiving blood 308 procured using the lancet or lancet device 302 (this collection container 306 can be a filter paper or other dry material for absorbing the blood 308), a storage device 310 for sealing the collection container 306 containing the blood 308 (pre-labeled with a bar code or QR code 312 designating the individual client and other related information), and a pre-labeled shipping package (envelope or box) 314 for return to the company for further processing. The lancet kit 300 can also contain a set of instructions 316 on how to use the lancet or lancet device 302 and how to return the blood sample 308 to the company.

A method of using the lancet kit 300 comprises the following steps: a) washing the skin of a fingertip or other body area using the isopropyl alcohol wipe or pad 304; b) puncturing the skin using the lancet or automated lancet device 302, allowing a drop of capillary blood 308 to form; c) depositing the blood 308 onto the collection container 306 (filter paper or other solid template material where the blood 308 will be absorbed); d) after the blood 308 dries on the collection container 306, placing the blood sample 308/collection container 306 in the pre-labeled storage device 310; and e) mailing the storage device 310 back to the company using the pre-paid, pre-labeled shipping package (envelope or box) 314.

Upon ordering either or both kits 100, 300, the client/individual will pre-register and set up a confidential portal with contact information. When the client receives the kit 100, 300, the client will also receive the storage device 108/112, 310/314 for returning the sample with its bar code 110, 312 that is unique to the client and containing their name, contact, and other information. Return shipping materials 112, 314 will be pre-paid and pre-labeled with a printed shipping address for returning the sample to the company. Once analyzed, the results can be put up on the client's portal so they will have them quickly, along with any company analysis.

The autophagy markers can be measured using a variety of techniques which include, but are not limited to: western blot, immunoblotting, automated fluorescence microscopy, mass-spectroscopy, mass-spectroscopy combined with chromatographic techniques, mRNA or other nucleic acid detection techniques, image-based cell counting, flow cytometry, and other appropriate methods as may be devised for this application.

A western blot (limited to use with the buccal kit 100) is a laboratory method used to detect specific protein molecules from among a mixture of proteins. This mixture can include all of the proteins associated with a particular tissue or cell type. Western blots can also be used to evaluate the size of a protein of interest, and to measure the amount of protein expression. Quantification by western blotting will involve detection and quantification of the autophagy markers p62 and LC3-II or other appropriate markers or autophagic flux. Data will be normalized against the level of tubulin or another common cellular marker.

The first step in a western blot is to prepare the protein sample by mixing it with a detergent (e.g., sodium dodecyl sulfate), which makes the proteins unfold into linear chains and coats them with a negative charge. Next, the protein molecules are separated according to their sizes using a method called gel electrophoresis. Following separation, the proteins are transferred from the gel onto a blotting membrane.

Once the transfer is complete, the membrane carries all the protein bands originally on the gel. Next, the membrane goes through a treatment called blocking, which prevents any nonspecific reactions from occurring. The membrane is then incubated with an antibody called the primary antibody, which specifically binds to the protein of interest. Following incubation, any unbound primary antibody is washed away, and the membrane is incubated yet again, but this time with a secondary antibody that specifically recognizes and binds to the primary antibody. The secondary antibody is linked to a reporter enzyme that produces color or light, which allows it to be easily detected and imaged. These steps permit a specific protein to be detected from among a mixture of proteins. Autophagy marker proteins will be normalized and reported as a function of a cell marker (or other method of cell counting) to determine the extent of autophagy activity based on the number of cells obtained in the sample.

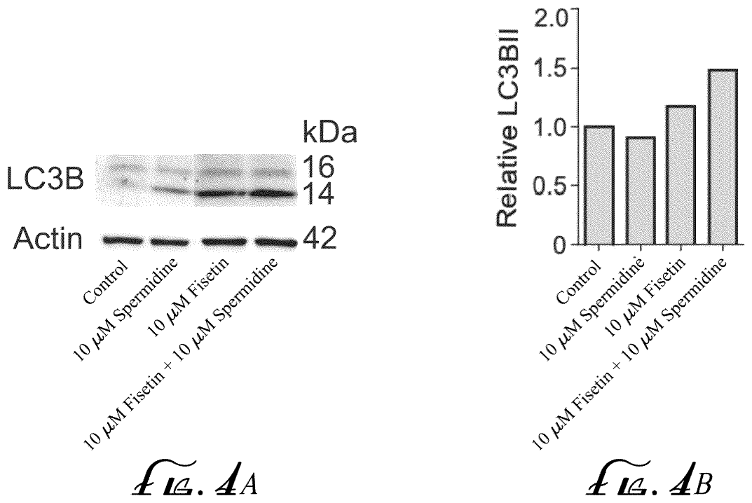

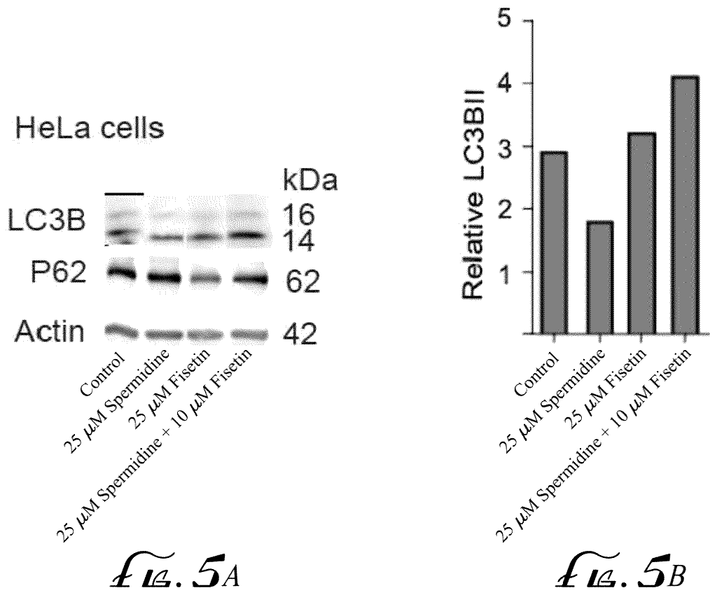

An example of the results of the western blot technique can be seen in FIGS. 4A, 4B, 5A, and 5B.

Chromatographic techniques may include immunoaffinity chromatography, in which a column containing antibodies for the desired markers are present, thus allowing for retention of only the desired markers. These markers may then be eluded from the column and further separated and detected using, for example, high-performance liquid chromatography and/or mass spectroscopy.

Markers may also be quantified using liquid chromatography paired with mass spectroscopy (LC-MS), in which the protein mixture is separated chromatographically, followed by identification of markers by mass spectroscopy. Specific techniques include but are not limited to liquid chromatography/triple quadrupolar mass spectroscopy (LC-MS/MS), liquid chromatography/high resolution mass spectroscopy (LC-HRMS), liquid chromatography/matrix assisted laser desorption/ionization-time of flight (LC-MALDI-TOF), or any combination thereof.

Specific messenger RNA related to autophagy stimulation and/or flux is another possible marker. Detection techniques include, but are not limited to, northern analysis, nuclease protection assays, and polymerase chain reaction (including RT-PCR and QPCR). Additionally, chromatography and mass spectroscopy may be applied to mRNA detection.

Microtubule associated protein 1A/1B—light chain 3 (LC3) is a marker protein involved in the formation of autophagosomes and autolysosomes, which are usually characterized and monitored by fluorescence microscopy using fluorescent protein-tagged LC3 probes (FP-LC3). The autophagy puncta are counted using automated microscopic techniques. This constitutes a production-line method, e.g., image-based cell counters that can utilize bright-field or fluorescent microscopes coupled with digital cameras (CMOS or CCD) to obtain images that are then analyzed with image analysis software. These automated cell counters are either self-contained (internal PC) or connected to an external computer. The sample is contained in a disposable, consumable (slide or cartridge) that ensures the same volume is analyzed each time, allowing for accurate volumetric counts. The consumable is an integral part of the counting system and its performance impacts accuracy of the results. Bright-field-based cell counters use colorimetric dyes (for example, Trypan blue) for cell viability analysis, whereas fluorescent cell counters use fluorescent dyes for viability analysis.

Flow cytometers are used to measure the characteristics of individual cells and particles that are delivered in a flow system past a point of measurement where light is focused on one cell or one particle at a time. Then, the scattered light and fluorescent signals of different wavelengths are recorded. Some flow cytometers use accurate volumetric pumps and are able to provide volumetric counts. To get absolute counts using cytometers that do not use the volumetric pumps, users have to introduce a calibrated means (typically beads) to determine the volumetric count.

Numerous fluorescent methods could be adapted to perform mass screening of autophagy markers in conjunction with the techniques described above or as may be derived independently.

With respect to the buccal kit 100, using the material extracted from an individual's buccal swab 102 (containing mixed human epithelial cells and leukocytes (several types) the basal level of autophagy will be determined by measuring a selected marker using one or more of the methods described above.

With respect to the lancet kit 300, using material extracted from the individual's blood sample 308 on the collection container 306, the basal level of autophagy will be determined by measuring a selected marker using one or more of the methods described above.

Once this level of initial autophagy activity is determined, a custom combination of autophagy stimulants (Autophagy Combination Product, ACP) will be selected for the client's treatment and sent to them with instructions for use.

The ACP formula is pre-determined based on in vitro experiments measuring the relative potency of several autophagy stimulants and synergistic/additive combinations thereof using one or more autophagy reporter cell lines (e.g., HeLa). Drug combinations are rationally selected based on their individual mechanisms of action and how they differ and complement each other in stimulating autophagic flux by interacting at different points in the autophagy pathway. In this way, maximum stimulation is achieved with minimum side effects.

Depending on the basal level of autophagy in an individual, either a standardized or custom combination of autophagy stimulants (ACP) can be designed for them.

The combination of autophagy stimulating compounds (ACP) can comprise, but is not limited to, one or more of the following compounds or related natural product extracts (i.e. extracts or dry materials containing the compounds(s)): resveratrol, curcumin/turmeric, spermidine, fisetin, beberine, quercetin, ginger, rutin, black pepper, oridonin, evodiamine, epigallocatechin gallate (EGCG), theanine, citrus bergamont, herbs (for example but not limited to, oregano, sage, or rosemary, etc.), isoliquiritigenin, alpha-magostin, wogonin, boswellia, leuteolin, piperlongumine, pterostilbene, wheat germ extract, bladderwrack extract, ginseng, anise, minerals (for example but not limited to zinc, manganese, etc.), grape seed extract, R-alpha lipoic acid, soy lipid extract, trehalose, caffeic acid, and salicin.

The ACP may also be combined with inert excipients or other compounds to improve absorption or other biological properties. Examples of inert excipients can include but are not limited to sucrose, mannitol, citric acid, ascorbic acid, cellulose, xylitol, poly(ethylene glycol), etc.

Prior to formula selection, compound and combination toxicities are evaluated. Examples of acceptable cytotoxic screening can include but are not limited to dye exclusion (e.g., Trypan blue, formazan), protease biomarkers, ATP content using CellTiter-Glo luminescent cell viability assay or another method.

After taking the selected ACP drugs for a pre-determined period of time (3 months or more, 6 months, 1 year, 2 years), the individual will undergo additional buccal swab/lancet testing to determine whether the individual's level of autophagy has changed.

The individual can remain on the ACP program, taking the selected compounds via a subscription that will automatically mail replacement compounds to the individual as needed, to ensure their autophagy levels remain elevated. This is ideal because failure to take the compounds could result in a decrease in autophagy levels and associated health benefits; therefore, providing these compounds to the individual via a subscription program that does not require them to remember to order refills is ideal. Shipments can include one or more buccal swab testing kits 100 or lancet kits 300 for monitoring autophagy activity during the course of the subscription.

Although ACP subscriptions will be optional, subscribers will be assigned a user interface/web portal by which the subscriber can monitor their own autophagy levels. Graphical analyses can be provided, comparing a baseline initial measurement with autophagy levels after prolonged use of the ACP stimulant combinations and/or lifestyle changes. Subscribers will be able to see applications of their data compared with the latest autophagy research as it applies to individual health. The site will also provide means to order autophagy test kits 100, 300 or additional ACP supplies. Individual counseling can also be provided.

The method of present invention is a method of stimulating autophagy in an individual by administering one or more compounds to the individual to stimulate autophagy. Optionally, the method comprises the additional steps of
a) providing the individual with a sample collection kit;
b) obtaining a sample from the individual;
c) testing the sample to determine the individual's level of autophagy; and then
d) administering one or more compounds to the individual to stimulate autophagy.

The sample collection kit can be a buccal swab kit 100 or a lancet kit 300. The buccal swab kit 100 comprises: i) at least one buccal swab 102; ii) at least one collection container 104; and iii) at least one pre-labeled storage device 108 wherein the storage device is pre-labeled with a bar code or QR code 110 designating the individual client and other related information, a pre-paid, pre-labeled shipping envelope or box 112 to return the storage device 108 containing the buccal swab 102. Optionally, the buccal swab kit 100 can include a set of instructions 106 on how to use the buccal swab 102 and at least one set of gloves (not shown).

The lancet kit 300 comprises a) at least one sterile lancet or an automated lancet device 302 for capillary bloodletting; b) a sealed packet with an isopropyl alcohol wipe or pad 304; c) a collection container 306 for receiving blood 308 procured using the lancet or lancet device 302; d) a storage device 310 for saving the collection container 306 containing the blood 308, wherein the storage device is pre-labeled with a bar code or QR code 312 designating the individual client and other related information; and e) a pre-labeled shipping package (envelope or box) 314 for return to the company for further processing. The lancet kit 300 can also contain a set of instructions 316 on how to use the lancet or lancet device 302 and how to return the sample 308 to the company. Optionally, the collection container 306 can be a paper filter or other solid material for absorbing the blood 308.

As noted above, the ACP compounds can comprise one or more of the following compounds or related natural product extracts or dry materials: resveratrol, curcumin/turmeric, spermidine, fisetin, beberine, quercetin, ginger, rutin, black pepper, oridonin, evodiamine, epigallocatechin gallate (EGCG), theanine, citrus bergamont, herbs (for example but not limited to, oregano, sage, rosemary, etc.), isoliquiritigenin, alpha-magostin, wogonin, boswellia, leuteolin, piperlongumine, pterostilbene, wheat germ extract, bladderwrack extract, ginseng, anise (for example but not limited to, Zn, Mn, etc.), grape seed extract, R-alpha lipoic acid, soy lipid extract, trehalose, caffeic acid, and salicin.

The step of administering can comprise administering one or more compounds orally or topically to the individual, and the compounds can be routinely delivered to the individual via a subscription program.

Optionally, the individual need not participate in the subscription program in order to utilize the compound(s).

### EXAMPLES

### Example 1: Determination of Autophagy Stimulation

Experimental details: Hela cells were seeded in 6 cm dishes (1 million cells/dish) 24h prior treatment, then treated the next day with different compounds along with the control solvent (DMSO). The compounds (spermidine, fisetin) were added both in the absence and presence of Bafilomycin A1 (100 ng/ml). Only the latter data is shown in FIGS. 4A-5B. Samples were harvested after 4 h incubation period.

Western blot protocol: Media was aspirated from the cell cultures, and the cells were then washed with 1×PBS. After washing, media was aspirated again and PBS was added again. Next, cells were scraped off the plate and transferred to a centrifuge tube, where they were centrifuged at 3000 rpm for 5 minutes. Next, the supernatant was discarded and the resulting pellet was dissolved in a lysis buffer containing a protease inhibitor. Then, the dissolved pellet was incubated in the lysis buffer for 30-45 minutes, and then centrifuged at 12000 rpm for 10 minutes. After centrifuging, the supernatant was transferred to a clean tube (membranes and cell debris remained in the pellet), and BCA (bicinchoninic acid assay) method was used to determine the protein concentration. Next, the samples were heated to 95-100° C. for 5 min and then allowed to cool. Next, the required amount of protein was loaded onto SDS-PAGE gel along with pre-stained molecular weight markers, which was then electro-transferred to a nitrocellulose membrane. The membrane was incubated in a blocking buffer for 1 hour at room temperature, then washed three times for 5 min each with 15 ml of TBST (a mixture of tris-buffered saline and polysorbate 20). Next, the membrane and primary antibody was incubated in 10 ml of primary antibody dilution buffer with gentle agitation overnight at 4° C. Next, the membrane was washed three times for 5 min each with 15 ml of TBST. Then, the membrane was incubated with HRP-linked or fluorescent dye conjugated with a secondary antibody in 10 ml of blocking buffer with gentle agitation for 1 hour at room temperature. Then, the membrane was washed three times for 5 min each with 15 ml of TBST, and the detection was undergone.

FIG. 4A depicts a western blot analysis of autophagy stimulation by spermidine (10 uM), fisetin (10 uM), and a combination of both compounds.

FIG. 4B is a graphical presentation of the western blot data in FIG. 4A.

FIG. 5A is a western blot analysis of autophagy stimulation by spermidine (25 uM), fisetin (25 uM), and a combination of both compounds.

FIG. 5B is a graphical presentation of the western blot data in FIG. 5A.

Conclusion: These ACP compounds function at different sites in the autophagy stimulation cascade. Spermidine acts at the elF5A level, increasing direct binding and promotion of translocation factor EB (TFEB) leading to the expression of autophagy genes. In contrast, fisetin acts at a different step, i.e., inhibition of mTOR, which then allows TFEB to be activated. These preliminary data indicate that the compounds have an additive or synergistic effect in stimulating autophagy which may be advantageous over products currently on the market.

The present invention has the following advantages:

It offers a simple and easy way to track autophagy levels in an individual and allow them to receive the compound(s) necessary to stimulate those levels if desired.

It offers a convenient, home-based measurement technique to allow an individual to monitor the efficacy of autophagy stimulants that act at different points in the autophagy cascade and to receive guidance from autophagy experts through their customized portal.

Health-conscious individuals can subscribe to a long-term monitoring program to receive autophagy stimulants and measurement materials.

No physician authorization required.

Quick return of results and analysis.

Results available on digital devices.

Updates on autophagy research are delivered directly to the consumer/individual.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference.

Features of the invention include:
1. A buccal swab kit comprising:
   a) at least one buccal swab;
   b) at least one collection container and pre-labeled storage device;
   c) at least one pre-labeled, pre-paid shipping package (envelope or box);
   d) a set of instructions on how to use the buccal swab; and
   e) at least one set of gloves.
2. A system for stimulating autophagy, the system comprising:
   a) a buccal swab kit comprising:
      i) at least one buccal swab;
      ii) at least one collection container and pre-labeled storage device;
      iii) at least one pre-labeled, pre-paid shipping package (envelope or box);
      iv) a set of instructions on how to use the buccal swab; and
      v) at least one set of gloves; and
   b) one or more compounds that stimulate autophagy.
3. The system of feature 2, whereby the ACP compounds can comprise at least two of the following non-proprietary drugs or other autophagy stimulants, including related natural product extracts or dry materials: resveratrol, curcumin/turmeric, spermidine, fisetin, beberine, quercetin, ginger, rutin, black pepper, oridonin, evodiamine, epigallocatechin gallate (EGCG), theanine, citrus bergamont, herbs (for example but not limited to, oregano, sage, rosemary, etc.), isoliquiritigenin, alpha-magostin, wogonin, boswellia, leuteolin, piperlongumine, pterostilbene, wheat germ extract, bladderwrack extract, ginseng, anise, minerals (for example but not limited to zinc, manganese, etc.), grape seed extract, R-alpha lipoic acid, soy lipid extract, trehalose, caffeic acid, and salicin.
4. A method of stimulating autophagy in an individual, the method comprising the steps of:
   a) providing the individual with a buccal swab kit, the buccal swab kit comprising:
      i) at least one buccal swab;
      ii) at least one collection container and pre-labeled storage device;
      iii) at least one pre-labeled, pre-paid shipping package (envelope or box);
      iv) a set of instructions on how to use the buccal swab; and
      v) at least one set of gloves;
   b) testing a buccal swab sample provided by the individual to determine the individuals level of autophagy; and
   c) administering one or more compounds to stimulate autophagy.
5. The method of feature 4, wherein the compounds of step c), the ACP compounds, can comprise at least two of the following non-proprietary drugs or other autophagy stimulants, including related natural product extracts or dry materials: resveratrol, curcumin/turmeric, spermidine, fisetin, beberine, quercetin, ginger, rutin, black pepper, oridonin, evodiamine, epigallocatechin gallate (EGCG), theanine, citrus bergamont, herbs (for example but not limited to, oregano, sage, rosemary, etc.), isoliquiritigenin, alpha-magostin, wogonin, boswellia, leuteolin, piperlongumine, pterostilbene, wheat germ extract, bladderwrack extract, ginseng, anise, minerals (for example but not limited to zinc, manganese, etc.), grape seed extract, R-alpha lipoic acid, soy lipid extract, trehalose, caffeic acid, and salicin.
6. The method of feature 4, wherein the step of administering comprises administering the compounds via a subscription program.
7. Compounds for stimulating autophagy, the ACP compounds can comprise at least two of the following non-proprietary drugs or other autophagy stimulants, including related natural product extracts or dry materials: resveratrol, curcumin/turmeric, spermidine, fisetin, beberine, quercetin, ginger, rutin, black pepper, oridonin, evodiamine, epigallocatechin gallate (EGCG), theanine, citrus bergamont, herbs (for example but not limited to oregano, sage, rosemary, etc.), isoliquiritigenin, alpha-magostin, wogonin, boswellia, leuteolin, piperlongumine, pterostilbene, wheat germ extract, bladderwrack extract, ginseng, anise, minerals (for example but not limited to Zn, Mn, etc.), grape seed extract, R-alpha lipoic acid, soy lipid extract, trehalose, caffeic acid, and salicin.

1. A system for stimulating autophagy, the system comprising:
a) a home-based sample collection kit comprising at least one buccal swab; and
b) one or more compounds that stimulate autophagy.

2. The system of claim 1, wherein the sample collection kit further comprises:
a) at least one collection container;
b) at least one pre-labeled storage device;
c) at least one pre-labeled, pre-paid shipping package;
d) a set of instructions on how to use the buccal swab; and
e) at least one set of gloves.

3-6. (canceled)

7. The system of claim 1, wherein the one or more compounds are selected from the group consisting of: spermidine, fisetin, beberine, ginger, black pepper, oridonin, evodiamine, epigallocatechin gallate (EGCG), theanine, citrus bergamont, herbs, oregano, sage, rosemary, isoliquiritigenin, alpha-magostin, wogonin, boswellia, leuteolin, piperlongumine, pterostilbene, wheat germ extract, bladderwrack extract, anise, minerals, zinc, manganese, grape seed extract, soy lipid extract, caffeic acid, and salicin.

8. The system of claim 7, wherein the one or more compounds are spermidine, fisetin and caffeic acid.

9. The system of claim 7, wherein the one or more compounds is spermidine.

10. A method of stimulating autophagy in an individual, the method comprising the steps of:
a) providing the individual with at least one sample collection kit to collect a sample;
b) testing the sample provided by the individual to determine a level of autophagy in the individual;
c) customizing a formula of one or more compounds for stimulating autophagy in the individual based on the level of autophagy in the individual; and
d) administering the formula to the individual to stimulate autophagy.

11. The method of claim 10, wherein the sample collection kit is a buccal swab kit that comprises:
a) at least one buccal swab;
b) at least one collection container;
c) at least one pre-labeled storage device;
c) at least one pre-labeled, pre-paid shipping package;
d) a set of instructions on how to use the buccal swab; and
e) at least one set of gloves.

12-15. (canceled)

16. The method of claim 10, wherein the one or more compounds are selected from the group consisting of: spermidine, fisetin, beberine, ginger, black pepper, oridonin, evodiamine, epigallocatechin gallate (EGCG), theanine, citrus bergamont, herbs, oregano, sage, rosemary, isoliquiritigenin, alpha-magostin, wogonin, boswellia, leuteolin, piperlongumine, pterostilbene, wheat germ extract, bladderwrack extract, anise, minerals, zinc, manganese, grape seed extract, soy lipid extract, caffeic acid, and salicin.

17. The method of claim 16, wherein the one or more compounds are spermidine, fisetin and caffeic acid.

18. The method of claim 17, wherein the one or more compounds is spermidine.

19. The method of claim 10, wherein the step of administering comprises administering the compounds via a subscription program.

20. A combination of compounds for stimulating autophagy, wherein the combination comprises two or more compounds selected from the group consisting of: spermidine, fisetin, beberine, ginger, black pepper, oridonin, evodiamine, epigallocatechin gallate (EGCG), theanine, citrus bergamont, herbs, oregano, sage, rosemary, isoliquiritigenin, alpha-magostin, wogonin, boswellia, leuteolin, piperlongumine, pterostilbene, wheat germ extract, bladderwrack extract, anise, minerals, zinc manganese, grape seed extract, soy lipid extract, caffeic acid, and salicin.

21. The method of claim 10, wherein testing the sample comprises measuring one or more markers selected from the group consisting of p62, LC3-II, and microtubule associated protein 1A/1B-light chain 3.

22. The method of claim 21, wherein the one or more markers is normalized against a level of tubulin.

23. The system of claim 1, wherein the one or more compounds are spermidine, resveratrol, and luteolin.

24. The system of claim 1, wherein the one or more compounds are trehalose, resveratrol, and luteolin.

25. The system of claim 1, wherein the one or more compounds are curcumin, trehalose, and piperlongumine.

26. The system of claim 1, wherein the one or more compounds are spermidine, caffeic acid, and piperlongumine.

27. The system of claim 1, wherein the one or more compounds are fisetin, curcumin, and resveratrol.

28. The system of claim 1, wherein the one or more compounds are curcumin and quercetin.

29. The system of claim 1, wherein the one or more compounds are fisetin and wogonin.

30. The system of claim 1, wherein the one or more compounds are spermidine and caffeic acid.
